(19) United States
(12) Patent Application Publication
(10) Pub. No.: US 2002/0005934 A1
(43) Pub. Date: Jan. 17, 2002

(54) Device for testing visual functions of the human eye

(75) Inventors: Hansueli Walther, Baden (CH); Beat Gisler, Baar (CH)

Correspondence Address: William H. Logsdon, 700 Koppers Building, 436 Seventh Avenue, Pittsburgh, PA, 15219-1818, US

(73) Assignee: INTERZEAG AG

(21) Appl. No.: 09/903,956

(22) Filed: Jul. 12, 2001

(30) Foreign Application Priority Data
Jul. 13, 2000 (EP) 00 810 615.5

(51) Int. Cl. A61B 3/10

(52) U.S. Cl. 351/221

(57) ABSTRACT

For purposes of examination, the human eye at a defined viewing point is oriented along a viewing axis. Using means associated with a first light source, stimuli are generated at selectable locations on the background of the viewing axis. Using a second light source, a homogeneous background light is generated around the viewing axis. The second light source has at least one LED. This LED can be actuated as required so that it generates a constant background light or a flickering background light. If at least two LEDs are used, the one LED can generate a colored background light by means of a fixed colored filter. No moving parts are necessary for the generation of a flickering background light, nor any means for the active cooling of the second light source.

### BACKGROUND OF THE INVENTION

1. Field of the Invention

This invention relates to a device for the examination of visual functions of the human eye which is at a viewing point and is oriented along a viewing axis. Means associated with a first light source are used for the temporally staggered generation of stimuli at selectable locations on the background of the viewing axis. The background around the viewing axis is uniformly illuminated with a second light source.

2. Description of the Related Art

A device of the prior art of the type described above is described in CH-A-677 599. In this device of the prior art, the stimuli are offered to the patient on an image plane around the viewing axis. The three-dimensional background around this viewing axis is uniformly illuminated. For this purpose, in the viewing axis there is a partly light-permeable mirror that is directed toward the image plane, which on one hand lets through the light of the stimulus light source and on the other hand deflects the light generated by the second light source toward the image plane. The second light source used is a halogen lamp which is optionally cooled by means of a ventilator.

The prior art also includes a full-field perimeter in the field of electrophysiology by means of which a flickering background can be generated. The flickering background is generated by means of a halogen lamp and a mechanical shutter which interrupts the light of the halogen lamp at a defined frequency. A mechanical shutter is relatively complex and expensive and is also prone to defects and malfunctions. SUMMARY OF THE INVENTION

The invention relates to a device for the examination of visual functions of the human eye which is at a viewing point and is oriented along a viewing axis. A first light source is equipped for the temporally staggered generation of stimuli at selectable locations on the background of the viewing axis. A second light source generates light that is distributed evenly on the background of the viewing axis.

### BRIEF DESCRIPTION OF THE DRAWINGS

FIG. 1 is a schematic illustration of the device of the present invention; and

FIG. 2 is a schematic illustration of an alternate embodiment of a second light source.

### DESCRIPTION OF THE PREFERRED EMBODIMENTS

The object of the invention is to create a device for testing visual functions of the human eye which can be manufactured more easily and economically.

The invention teaches that the second light source has at least one LED (Light Emitting Diode). Associated with the LED there are preferably timing elements that allow the LED to be turned on either constantly or to flicker at selectable timed ratios. The frequencies that can be selected are essentially unlimited and thus cover even the ranges in which the critical flicker fusion frequencies occur.

The device claimed by the invention has the advantage that there is no need for moving mechanical parts to generate the flickering background. LEDs also generate no waste heat, which means that active cooling by means of a ventilator is unnecessary. In contrast to electrophysiology, where expensive amplifiers are necessary to evaluate the flicker ERGs, the subject responds by pressing a response button.

In one development of the invention, the second light source has at least two LEDs that are located in different planes and at least one colored filter. The light from the one LED thereby passes through the colored filter and generates a correspondingly colored background. In this case, the essential advantage is that the colored filter can be installed in a stationary manner. In the prior art, such colored filters had to be mounted so that they were movable and could be pivoted into the beam path when necessary. In this invention, to generate a white background, the LED, the light from which does not go through the colored filter, is turned on. To generate a colored background, the other LED is turned on. In this case, realizations with a plurality of colored filters are possible.

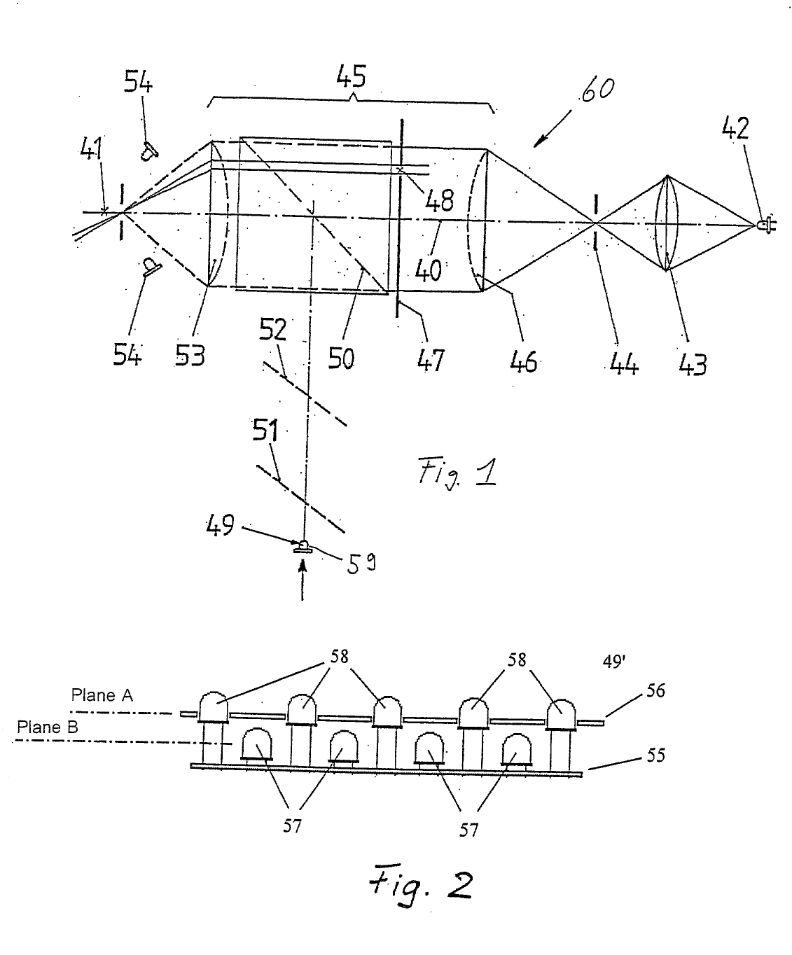

The perimeter that is illustrated schematically in FIG. 1 has an optical system 60, on the optical axis 40 of which there are on one hand a viewing point 41 and on the other hand a first light source 42 to generate stimuli. The stimuli are brief pulses of light that are presented to the subject at different points in the subject's field of vision. The light from the first light source 42 is focused by a condenser lens 43 on a diaphragm 44 and is deflected behind the diaphragm 44 into a parallel beam path 45. The beam path 45 is limited toward the diaphragm 44 by a field lens 46 and toward the viewing point 41 by a convex lens 53. The viewing point 41 is then realized so that the convex lens 53 focuses the light that exits the beam path 45 onto the center of the pupil of the subject's eye. Sight defects of the subject's eye therefore have no influence on the stimulus image on the retina. In the parallel beam path 45, perpendicular to the viewing axis 40, there is a diaphragm 47 which blocks the entire cross section, with a diaphragm opening 48 which limits the stimulus in terms of shape and size. The diaphragm 47 can be displaced in a plane in two directions that are oriented perpendicular to each other with respect to viewing axis 40 by means of a drive which is not shown here and is controlled by a computer which is also not shown here. Consequently, the diaphragm opening 48 can be displaced by said drive to any desired location in the diaphragm plane and defined in terms of coordinates. The stimuli are presented in the parallel beam path 45 in the image plane, from which they are projected by the convex lens 53 onto the retina of the subject's eye.

The background is illuminated by means of a second light source 49, the light from which is deflected by means of a partly light-permeable deflecting mirror 50 in the beam path 45 toward the convex lens 53. By means of two additional partly light-permeable deflecting mirrors 51 and 52, a fixation mark can be superimposed onto the background on one hand and the subject's eye is monitored by means of a camera on the other hand.

The second light source 49 used is an LED that is actuated so that on one hand a continuous background light and on the other hand a flickering background light can be generated. Programming strategies can be used to modify both the intensity and the characteristic of the flickering background light, so that the critical flicker fusion frequency can be determined by means of calibration methods based on bracketing or by continuous approximation. The perception of the flickering background is indicated by the subject pushing a response button. The background light can be white or colored. In FIG. 1, the second light source 49 is formed by a single LED. However, realizations are also possible in which the second light source 49 is formed by a plurality of LEDs.

In the realization of a second light source 49′ illustrated in FIG. 2, a plurality of LEDs 57 and 58 are located on a bracket 55 in different planes A and B. A colored filter 56 is fastened to the bracket so that the LEDs 58 are located above this filter and the LEDs 57 are located below this filter 56. To generate white background light, only the LEDs 58 are operated. To obtain colored background light, the LEDs 57 but not the LEDs 58, are operated. Because the LEDs 57 are located below the colored filter 56, as shown in the accompanying illustration, their light falls through this colored filter 56 and thereby generates the corresponding color. To change the color, all that is necessary is to reverse the switching of the LEDs 57 and 58. The colored filter 56 can be installed in a stationary manner and therefore need not be moved to change the color. Because the LEDs 57-59 emit essentially no heat during operation, there is need for active cooling of the second light source 49 or 49′.

What is claimed is:

1. A device for testing visual functions of the human eye which is at a defined viewing point and oriented along a viewing axis, comprising means associated with a first light source for the temporally staggered generation of stimuli at selectable locations on the background of the viewing axis and with a second light source for the generation of light that is distributed evenly on the background of the viewing axis, wherein the second light source has at least one LED.

2. The device as claimed in claim 1, wherein the second light source is equipped so that a constant background light or a flickering background light can be generated as desired.

3. The device as claimed in claim 2, wherein the frequency of the flickering background light can be modified at least in the range of conventional critical flicker fusion frequencies.

4. The device as claimed in claim 2, wherein the intensity of the flickering background light can be modified.

5. The device as claimed in claim 1, wherein the second light source has at least two LEDs and at least one colored filter, whereby the light of the first LED goes through the colored filter and generates a corresponding colored background light.

6. The device as claimed in claim 5, wherein one LED is located below the colored filter and the other LED is located above the colored filter.

7. The device as claimed in claim 1, wherein a plurality of LEDs generate a colored background light by means of a colored filter.
